(19) United States
(12) Patent Application Publication
(10) Pub. No.: US 2010/0224197 A1
(43) Pub. Date: Sep. 9, 2010

(54) SLEEP APPLIANCE

(76) Inventor: Bryan Keropian, Tarzana, CA (US)

Correspondence Address: LEWIS, BRISBOIS, BISGAARD & SMITH LLP, 221 NORTH FIGUEROA STREET, SUITE 1200, LOS ANGELES, CA, 90012, US

(21) Appl. No.: 12/161,794

(22) PCT Filed: Feb. 1, 2007
(86) PCT No.: PCT/US07/02761

§ 371 Date: Jul. 22, 2008

Related U.S. Application Data
(60) Provisional application No. 60/764,435, filed on Feb. 2, 2006.

(51) Int. Cl. A61F 5/56

(52) U.S. Cl. 128/848

(57) ABSTRACT

A device for reducing snoring and/or sleep apnea comprising, in combination, a dental oral appliance, removably attached to a continuous positive airway pressure machine, to open the airway for a sleeping individual who suffers with snoring or obstructive sleep apnea, said dental oral appliance comprising, a body portion covering the inside of the upper teeth, said body having an open-palate, means to removably affix the appliance to the upper teeth, means to prevent occlusion of the upper and lower teeth, and a transpalatal bar that extends from the inside of the right teeth to the inside of the left teeth, to inhibit and restrain the tongue from upward and backward movement.

### TECHNICAL FIELD

This invention relates to a combination of a continuous positive airway pressure machine and an intra-oral device for reducing or eliminating snoring and/or sleep apnea.

### BACKGROUND ART

It has been estimated that ninety million American adults and children snore and that one in every ten adults snores. Snoring can have serious medical consequences for some people. Snoring is the first indication of a potentially life-threatening sleep disorder called Obstructive Sleep Apnea. If not diagnosed or if left untreated, Obstructive Sleep Apnea could result in severe medical consequences such as systemic high blood pressure, cardiovascular disease and even sudden death.

Snoring is caused by vibration of the tissues due to air turbulence as the airway narrows and may be a sign that a patient is suffering from apnea. But not all snorers suffer from apnea. Snoring can be categorized by its severity. There is the snorer who snores but experiences no physical problems. Then, there is the snorer who suffers from apnea, or the snorer who suffers from upper airway resistance. In some of these people, though they may not actually experience apneic episodes, their snoring is so loud and their breathing so labored, that it still wakes them, and their partners, numerous times throughout the night.

Many spouses, partners and/or children suffer through the night from the annoying noise of the snorer. Snoring not only disturbs the sleeping pattern of the snorer himself, it is also disruptive to the family life by causing lack of sleep to all involved. This leaves all involved unrefreshed, tired and sleepy throughout the day. It can cause sleepiness while driving, reading, working or doing other tasks.

A broad variety of intra-oral and dental appliances and devices are now available to treat a patient for snoring. Some known oral devices for treating snoring and obstructive sleep apnea are worn inside of the mouth and work by repositioning of the jaw, moving the mandible, lifting the soft palate or moving the tongue forward.

Other treatments for snoring include various surgeries, which are drastic steps to take to attempt to cure the problem, however snoring can be so disruptive to a person's life and relationships, that some sufferers resort to surgery.

Another method used to treat snoring includes the use of controlled positive air-flow pressure systems also known as CPAP, which require a nose mask and which are quite uncomfortable to use.

To understand the nature of the snoring and sleep apnea problem, the mechanism of snoring and obstructive sleep apnea must be understood. While we sleep, the tongue falls back and up towards the palate and it partially or completely obstructs or closes the airway path. This results in snoring, obstructive sleep apnea, or Upper Airway Resistance Syndrome. The medical treatment for these maladies range from medication to a CPAP (Continuous Positive Airway Pressure) machine. The CPAP is nearly 100% successful when utilized. Unfortunately, the non-compliance for CPAP use ranges from 50% to 80% depending where one searches in the literature.

### DISCLOSURE OF THE INVENTION

The sleep appliance method described herein includes all of the embodiments of the intra-oral devices described in my U.S. Pat. No. 6,766,802, issued on Jul. 27, 2004 and my pending U.S. application Ser. No. 11/165,641, filed Jun. 23, 2005, which are incorporated herein by reference, as though set forth fully herein.

The sleep appliance of this invention is a dental oral appliance for use with patients who suffer with sleep disorders and use a continuous positive air pressure machine (CPAP machine) as a treatment for their disorders. Primarily it is designed to reduce or eliminate snoring and to open the airway for a sleeping individual who suffers with obstructive sleep apnea.

The majority of snoring/sleep apnea patients cannot tolerate the uncomfortable straps and headgear that are required be worn when using a CPAP machine. One of the big complaints by users of a CPAP is, “it is like sleeping with a leaf blower in your face”. The air pressure provided by the CPAP is to push the tongue blocking the airway out of the way and allow air to reach the lungs.

Applicant's invention comprises a mouthpiece that allows easier compliance, since the irritating straps, headgear, or mask, are eliminated. The intra-oral device or mouthpiece is easy to use and fits to the patient's upper teeth (upper arch). This allows all nasal components to remain in absolute alignment regardless of a patient's sleep movements.

The sleep appliance of this invention, when used in combination with a CPAP machine, is designed to keep open the airway for a sleeping individual who suffers with snoring or obstructive sleep apnea.

As described in my issued patent and pending application referred to above, one embodiment of the mouthpiece appliance is physically designed similar to an upper (maxillary) orthodontic retainer, commonly called a Hawley retainer. It covers the inside (lingual) of the upper teeth and has an open palate (nothing covering the middle area of the palate). The body of the appliance has a series of recesses to fit against the lingual side of the teeth. Retention (holding ability) for the appliance is provided by acrylic fittings which hold the appliance in place in the same manner as an occlusal night guard.

In one embodiment, the anterior area has a raised strip or ramp that extends from the incisal tip (biting edge) of two or more of the incisors toward the lingual. It extends back a short distance from the incisors (where they meet or touch each other). This raised anterior strip acts as a bite discluder, to disclude or separate the posterior teeth.

In another embodiment there is no anterior ramp. The upper and lower teeth are separated by raised posterior ramps. This embodiment allows more room for the tongue to come forward.

There is a transverse strip, a transpalatal bar, that extends from the inside (lingual) of the upper right molars to the inside of the upper left molars. This transverse bar extends from the right to the left and covers the tongue. It does not touch the tongue unless the tongue attempts to move upward or backward, as often happens during sleep and causes snoring or sleep apnea. The transpalatal bar inhibits and restrains the upward and backward movement of the tongue, keeping the airway open during sleep.

Optionally, the transpalatal bar is slightly curved upward at its center, so that it does not touch the tongue but passes just over the tongue when the tongue is in its normal position. Also, the transpalatal bar does not touch the palate. By not touching either the tongue or the palate, the device of this invention is comfortable to wear and easily tolerated by patient users. Prior art devices which have either pushed the tongue down or pressed the palate up, were found to be unusable, as they often either created a gag reflex by the user or were so uncomfortable that they were unable to be tolerated by the user. The exact radius of the curvature of the transpalatal bar is determined by the physical dimensions and structure of each individual patient's anatomy. Some patients may need little or no curvature to achieve optimum results and other patients may need more curvature. The object is to have the transpalatal bar not touch the tongue or the palate, so that it will be tolerated, but inhibit and restrain any upward or backward movement of the tongue during sleep.

In addition, in the devices of this invention, there is optionally a posterior tongue restrainer (a tail) that extends backward from the center of the transpalatal bar. This posterior tongue restrainer provides a further barrier to the tongue's superior and posterior movement that blocks the airway to the posterior portion of the mouth. The posterior tongue restrainer, like the transpalatal bar, does not touch the tongue in its normal position but does restrain and inhibit the upward and backward movement of the tongue during sleep. This posterior tongue restrainer may be added to all of the appliances that are described in my issued patent and in my copending application.

The transpalatal bar may also have one or more bumps on the bottom, as described in my copending application, to further aid in restraining the upward and backward movement of the tongue.

Restraining the tongue results in major patient compliance improvement when using a CPAP. With the airway staying more open, due to the intra oral mouthpiece with the transpalatal bar, less air pressure is needed for the CPAP to perform its function. The “leaf blower in your face” is turned significantly down. This results in more comfort to the patient, and greater compliance with its use.

The CPAP is 100% successful when utilized. The problem, as described in the literature, is that 50%-80% of the patients who use the CPAP reject it. The rejection is because of discomfort. There are many reasons for the discomfort, as stated above. Applicant's design eliminates a number of the discomforting factors; no head straps to hold the apparatus in place, no mask, and a significant drop in the air pressure blowing in, to push the tongue out of the way.

Applicant's design incorporates alternate mouthpieces. One has the transpalatal bar as far posterior as it can be placed. A second design has the transpalatal bar forward of the first design, such as half-way back, that is, midway between the back of the front teeth and the back of the appliance. The second design is utilized because a number of patients find the posterior transpalatal bar confining. The transpalatal bar may be fixed in place anywhere between the back of the front teeth and as far back as it can be placed, depending upon the needs of the patient. By having the transpalatal bar adjustable as described, it allows the patient to use the design of the appliance, which is more comfortable.

The sleep appliance of this invention is designed to treat the problem of tongue blockage when sleeping. It works by utilizing several factors. First, it changes the vertical dimension (height of the opening or separation of the teeth). This results in an increased opening of the airway. Second, the transpalatal bar that runs transverse along the back of the appliance effectively inhibits and restrains any upward or backward movement of the tongue that would block the airway opening during sleep. Optionally, if needed, the sleep appliance can also include mandibular advancement to increase the opening, thus increasing the airway.

### OBJECTS OF THE INVENTION

It is an object of this invention to provide a simple device to make the use of a CPAP machine more comfortable and thus prevent or reduce snoring as well as Obstructive Sleep Apnea.

It is another object of this invention to provide a device, easily applied and easily tolerated, which will substantially increase compliance of patients using a CPAP machine.

Further objects and advantages will become apparent from a consideration of the following description and drawings.

### BRIEF DESCRIPTION OF THE DRAWINGS

FIG. 1 is a perspective view of the hose portion of a typical CPAP machine with Applicant's mouthpiece attached;

FIG. 2 is a bottom view of one embodiment of the intra-oral appliance;

FIG. 3 is a bottom view of another embodiment of the intra-oral appliance;

FIG. 4a is a bottom view of another embodiment of the intra-oral appliance; and,

FIG. 4b is a bottom view of another embodiment of the intra-oral appliance.

### BEST MODE FOR CARRYING OUT THE INVENTION

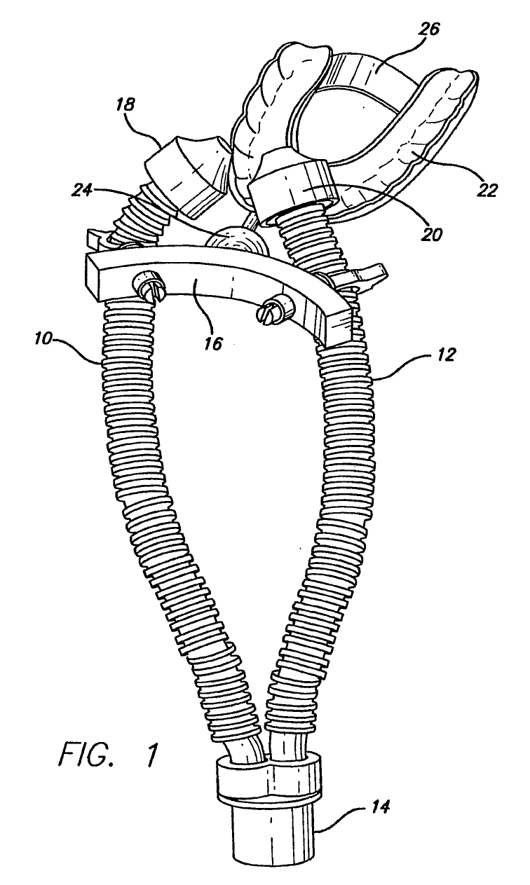

Referring now to FIG. 1 there is shown a pair of hoses 10 and 12 coming from a CPAP air pressure machine (not shown). The air pressure flow produced passes through a fitting 14, which divides the air flow into two hoses 10 and 12, one for each nostril.

Hoses 10 and 12 are held in place at their upper portion by a bracket 16, which places the nostril fittings 18 and 20 in the proper position to he inserted into the nostrils of the patient.

Attached to bracket 16 is the intra-oral appliance, mouthpiece 22, held in proper position by bracket 24. Mouthpiece 22 is placed into the patient's mouth and held against the patient's upper teeth as explained herein. No straps or mask are required. Mouthpiece 22 is removably attached to bracket 24, so that it may removed and cleaned.

Transpalatal bar 26 is connected across the rear portion of mouthpiece 22 and acts to inhibit and restrain the tongue from moving upward or backward during sleep, keeping the airway open.

Detailed descriptions of various embodiments of mouthpiece 22, are set forth in my U.S. Pat. No. 6,766,802 and my pending U.S. patent application Ser. No. 11/165,641, which descriptions are incorporated herein by reference. Transpalatal bar 26 may be straight or it may preferably be curved, depending upon the needs of the patient. It may pass across the rear portion of mouthpiece 22, as shown in FIG. 1, or may pass across near the back of the front teeth, or anywhere in between, depending upon the needs of the patient.

All of the devices described herein, which are totally or partially made of plastic, are preferably made of acrylic plastic or talon plastic.

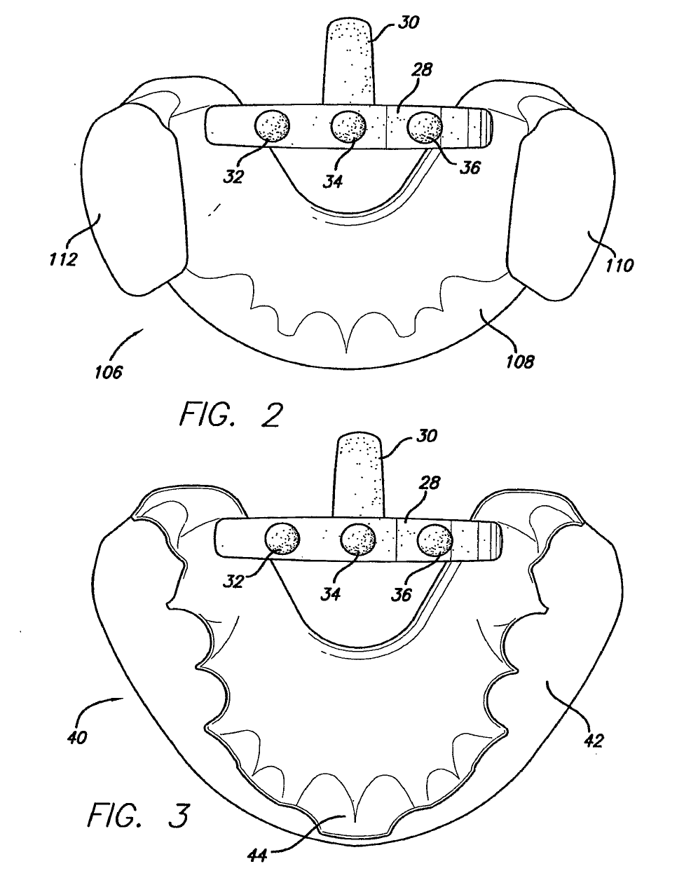

Referring now to FIG. 2 there is shown a first embodiment 106 of the sleep appliance of this invention comprising a body 108. Body 108 is made entirely of an acrylic plastic, commonly used for dental devices, and is custom fitted to fit over the wearer's posterior teeth in the same manner as an occlusal night guard, which uses an occlusal coverage. The occlusal coverage holds appliance 106 firmly onto the posterior teeth.

Raised posterior ramps 110 and 112 provide a surface against which the lower teeth occlude. Transpalatal bar 28 inhibits and restrains the tongue from moving upward or backward during sleep. Transpalatal bar 28 does not touch the tongue in its normal position, nor does transpalatal bar 28 touch the palate. However transpalatal bar 28 will prevent the tongue from moving upward or backward from its normal position. Optional posterior tongue restrainer 30 is attached to the center rear portion of transpalatal bar 28 and extends rearward to further inhibit and restrain the movement of the tongue. Posterior tongue restrainer 30 also does not touch the tongue in its normal position but may be needed for certain patients, to help restrain the tongue from upward or backward movement.

Optional posterior projections, 32, 34 and 36 are added to the bottom of transpalatal bar 28, if needed. They also do not touch the tongue in its normal position, but aid, if needed, in inhibiting and restraining the upward and backward movement of the tongue during sleep. Posterior projections 32, 34 and 36 may be from about 1 mm to about 6 mm long depending on the needs of the patient. While they are shown as cylindrical in shape, they may be any shape, such as rectangular, conical, oval, or any other shape.

Referring now to FIG. 3, there is shown another embodiment of the intra-oral appliance 40 having a body 42. Body 42 is made entirely of an acrylic plastic, commonly used for dental devices, and is custom fitted to fit over the wearer's posterior teeth in the same manner as an occlusal night guard, which uses an occlusal coverage. The occlusal coverage holds appliance 40 firmly onto the posterior teeth.

There is a raised anterior strip 44 that extends from the incisal tip (biting edge) of two or more of the incisors toward the lingual. Raised anterior strip 44 extends back a short distance from the central incisors towards the palate. Strip 44 acts as a bite discluder, separating the posterior teeth. Strip 44 is preferably from about 3 mm to about 5 mm thick in order to separate the posterior teeth.

Transpalatal bar 28 inhibits and restrains the upward and backward movement of the tongue to keep the airway open. Optional posterior tongue restrainer 30 is attached to the center rear portion of transpalatal bar 28 and extends rearward to further inhibit and restrain the upward and backward movement of the tongue during sleep. Optional posterior projections, 32, 34 and 36 are added to the bottom of transpalatal bar 28, if needed, as described above in FIG. 2.

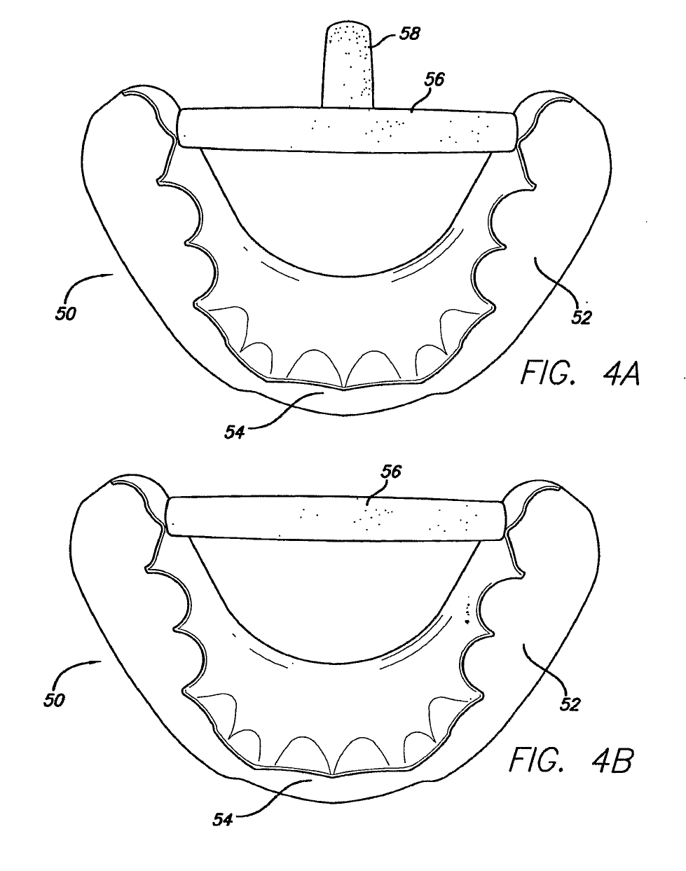

Referring to FIGS. 4A and 4B there is shown another embodiment of an appliance 50 with a body 52 and an anterior strip or ramp 54. Body 52 is made entirely of an acrylic plastic, commonly used for dental devices, and is custom fitted to fit over the wearer's upper posterior teeth in the same manner as an occlusal night guard, which uses an occlusal coverage.

Transpalatal bar 56 inhibits and restrains the upward and backward movement of the tongue to keep the airway open during sleep. Optional posterior tongue restrainer 58, in the device shown in FIG. 4A, optionally not on the device shown in FIG. 4B, is attached to the center rear portion of transpalatal bar 56 and extends rearward to further inhibit and restrain the upward and backward movement of the tongue.

Transpalatal bar 56 may be straight or curved, depending upon the needs of the patient. The presence or absence of the posterior tongue restrainer or the posterior projections, as well as the particular type of appliance from the various embodiments shown, is chosen based upon what works best for the individual patient.

As stated above, Applicant's design eliminates a number of the discomforting factors in the use of a CPAP machine; no head straps to hold the apparatus in place, no mask, and a significant drop in the air pressure blowing in, to push the tongue out of the way.

### INDUSTRIAL APPLICABILITY

The intra-oral device of this invention may be sold to any person who suffers from a snoring problem, or from obstructive sleep apnea, to make the use of a CPAP machine much more tolerable, to effectively reduce or eliminate snoring or sleep apnea.

Having thus described the invention, it is requested that the invention be described by the scope of the following claims.

1. A device for opening the airway for a sleeping individual who suffers with snoring or obstructive sleep apnea comprising, in combination, a continuous positive airway pressure machine and an intra-oral dental appliance to be worn by the sleeping individual comprising, a body portion covering the inside of the upper teeth, said body having an open palate, structure to removably affix the appliance to the upper teeth, structure to prevent occlusion of the upper and lower teeth, a transpalatal bar extending from the inside of the right teeth to the inside of the left teeth, to inhibit and restrain the upward and backward movement of the tongue and structure to removably attach the appliance to the continuous positive airway pressure machine.

2. The device of claim 1 in which the dental oral appliance transpalatal bar is curved.

3. The device of claim 1 in which the structure to removably affix the dental oral appliance to the upper teeth comprises an occlusal coverage.

4. The device of claim 1 in which the structure to prevent occlusion of the upper and lower teeth comprises a raised incisor ramp that extends from two or more incisors toward the lingual.

5. The device of claim 1 in which the structure of the dental oral appliance to prevent occlusion of the upper and lower teeth comprises raised posterior ramps.

6. The device of claim 1 in which the body of the dental oral appliance has a series of recesses to fit against the lingual side of the teeth.

7. The device of claim 1 in which the dental oral appliance is made of acrylic plastic.

8. The device of claim 1 in which a posterior tongue restrainer is attached to the transpalatal bar.

9. The device of claim 1 further comprising a plurality of raised projections on the bottom of the transpalatal bar.

10. A device comprising in combination, a dental oral appliance removably attached to a continuous positive airway pressure machine for reducing snoring and/or sleep apnea, said dental oral appliance comprising, a body portion covering the inside of the upper teeth, said body having an open palate, means to removably affix the appliance to the upper teeth, means to prevent occlusion of the upper and lower teeth, a transpalatal bar that extends from the inside of the upper right teeth to the inside of the upper left teeth, to inhibit and restrain the upward and backward movement of the tongue and means to removably attach the appliance to the continuous positive airway pressure machine.

11. The device of claim 10 in which the dental oral appliance transpalatal bar is curved.

12. The device of claim 10 in which the structure to removably affix the dental oral appliance to the upper teeth comprises an occlusal coverage.

13. The device of claim 10 in which the structure to prevent occlusion of the upper and lower teeth comprises a raised incisor ramp that extends from two or more incisors toward the lingual.

14. The device of claim 10 in which the structure of the dental oral appliance to prevent occlusion of the upper and lower teeth comprises raised posterior ramps.

15. The device of claim 10 in which the body of the dental oral appliance has a series of recesses to fit against the lingual side of the teeth.

16. The device of claim 10 in which the dental oral appliance is made of acrylic plastic.

17. The device of claim 10 in which a posterior tongue restrainer is attached to the transpalatal bar.

18. The device of claim 10 further comprising a plurality of raised projections on the bottom of the transpalatal bar.
